(19) United States
(12) Patent Application Publication
(10) Pub. No.: US 2010/0063591 A1
(43) Pub. Date: Mar. 11, 2010

(54) VERTEBRAL DISK PROSTHESIS AND SET OF ELEMENTS FOR THE CONSTITUTION OF A VERTEBRAL DISK PROSTHESIS

(75) Inventor: Mohan Emmanuel, Station Town Durham (GB)

Correspondence Address: YOUNG & THOMPSON, 209 Madison Street, Suite 500, Alexandria, VA, 22314, US

(73) Assignee: IMPLANTS INTERNATIONAL LIMITED, Cleveland Thornaby-on-tees (GB)

(21) Appl. No.: 12/373,940

(22) PCT Filed: Jun. 29, 2007
(86) PCT No.: PCT/IB07/01978

§ 371 Date: Aug. 31, 2009

(30) Foreign Application Priority Data
Jul. 18, 2006 (FR) 06 06504

(51) Int. Cl. A61F 2/44

(52) U.S. Cl. 623/17.15; 623/17.16

(57) ABSTRACT

A prosthesis includes: an upper plate; a lower plate (4); two assemblies forming a first installation element, wherein each is attached to the upper plate or the lower plate, both assemblies being arranged at a distance from each other in the frontal plane and substantially at equidistance from the median anteroposterior plane of the plate; and two elongated elastic rings, perpendicularly compressible to their lengths, and each including a second installation element on a longitudinal portion, each of these rings being intended to be installed on one of the assemblies forming the first installation element by the second installation element, being arranged substantially parallel with the anteroposterior plane of the prosthesis, and each of these rings being intended to bear against the lower plate and receive the upper plate.

The present invention relates to a vertebral disk prosthesis and a component assembly for the formation of a vertebral disk prosthesis.

A large number of vertebral disk prostheses exist. However, some existing prostheses have the drawback of not restoring the lordosis position of the vertebrae and/or the anatomical movement thereof perfectly. Other existing prostheses have the drawback of not being quickly and easily adaptable to the different situation liable to arise, particularly during the procedure; in this way, the same prosthesis may not be easily adapted to be either a “dynamic” prosthesis, i.e. enabling a movement of the vertebrae with some damping, or a prosthesis free from such damping.

The present invention is intended to remedy such drawbacks.

To this end, the prosthesis according to the invention comprises:
an upper plate, provided, on one side, with anchoring means to the lower side of the overlying vertebra to be treated;
a lower plate, provided, on one side, with anchoring means to the upper side of the underlying vertebra to be treated;
two assemblies forming first installation means, wherein each is attached to the upper plate or the lower plate, both assemblies being arranged at a distance from each other in the frontal plane and substantially at equidistance from the median anteroposterior plane of the plate; and
two elongated elastic rings, perpendicularly compressible to their lengths, and each comprising second installation means on a longitudinal portion, each of these rings being intended to be installed on one of said assemblies forming the first installation means by means of, said second installation means, being arranged substantially parallel with the anteroposterior plane of the prosthesis, and each of these rings being intended to bear against the lower plate and receive the upper plate.

In this way, the prosthesis according to the invention comprises two plates and two rings arranged in the anteroposterior plane, in parallel with each other and at a distance from the median anteroposterior plane of these vertebrae, these rings being elastic and compressible, and being inserted directly between the upper and lower plates.

These rings, in combination with said plates, enable a perfect restoration of the anatomical position of both treated vertebrae and a perfect restoration of the anatomical movement of said vertebrae, with a damping effect of the weight bearing thereon by the overlying vertebra.

In particular, the rings may be made of metal, particularly titanium alloy.

Preferentially, each ring comprising two longitudinal portions, at least one of the longitudinal portions of each ring is rounded in the frontal plane in order to enable a rolling movement on the ring of the plate against which said longitudinal section bears, according to the movement of the overlying vertebra with respect to the underlying vertebra.

The risk of metal particle generation due to friction is thus reduced to a minimum.

Each ring may have, viewed in the plane of the opening thereof, an elliptical shape; alternatively, it may display a plane longitudinal portion, either the lower portion or the upper portion.

According to a possible embodiment of the invention, said first installation means comprise an oval shaped block and said second installation means comprise a corresponding oval opening or cavity.

The installation of a ring on the plate is thus carried out by inserting a block of the plate or the ring in, respectively, a corresponding opening of the ring or the plate.

Preferentially, each block has a projecting peripheral rim at its free end, forming a retention means of the block in the corresponding opening or cavity.

The component assembly according to the invention comprises at least one upper plate, at least one lower plate and at least two rings such as those mentioned above, and at least one intermediate plate with a non-elastic structure, each intermediate plate having a convex rounded side and third installation means on the opposite side thereof, said third installation means being able to cooperate with said first installation means to enable an installation of said intermediate plate on either the upper or lower plate.

This component assembly makes it possible to form either a “dynamic” prosthesis comprising the two rings mentioned above, or a prosthesis with no damping effect comprising said intermediate plate, by simply adapting on one of the plates, either both rings, or the intermediate plate. This component assembly thus makes it possible to obtain a vertebral disk prosthesis perfectly adaptable to the different situations liable to arise, particularly during the procedure.

Preferentially, the component assembly comprises several upper plates and/or lower plates and/or several pairs of elastic rings and/or several intermediate plates of different sizes.

According to a preferred embodiment of the invention, said third installation means consist of a recess or blocks intended to cooperate with, respectively, blocks or corresponding cavities of one of the plates. When the blocks comprise the abovementioned projecting peripheral rim, the corresponding recess(es) comprise corresponding peripheral grooves, formed to enable click and ratchet of said rims together.

The invention will be understood more clearly and other characteristics and advantages will emerge, with reference to the schematic figure appended, representing, as non-limitative examples, several possible embodiments of components of the component assembly according to the invention.

For simplification purposes, the parts or components of an embodiment that are found in an identical or similar manner in another embodiment will be identified by the same numeric references and will not be described again.

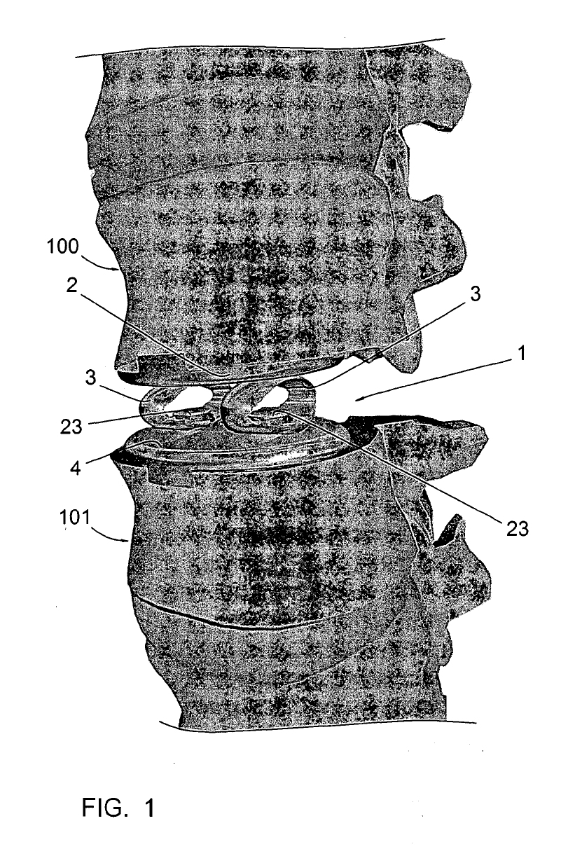

FIG. 1 is a view of a vertebral disk prosthesis consisting of components of said component assembly, while it is fitted between two vertebrae;

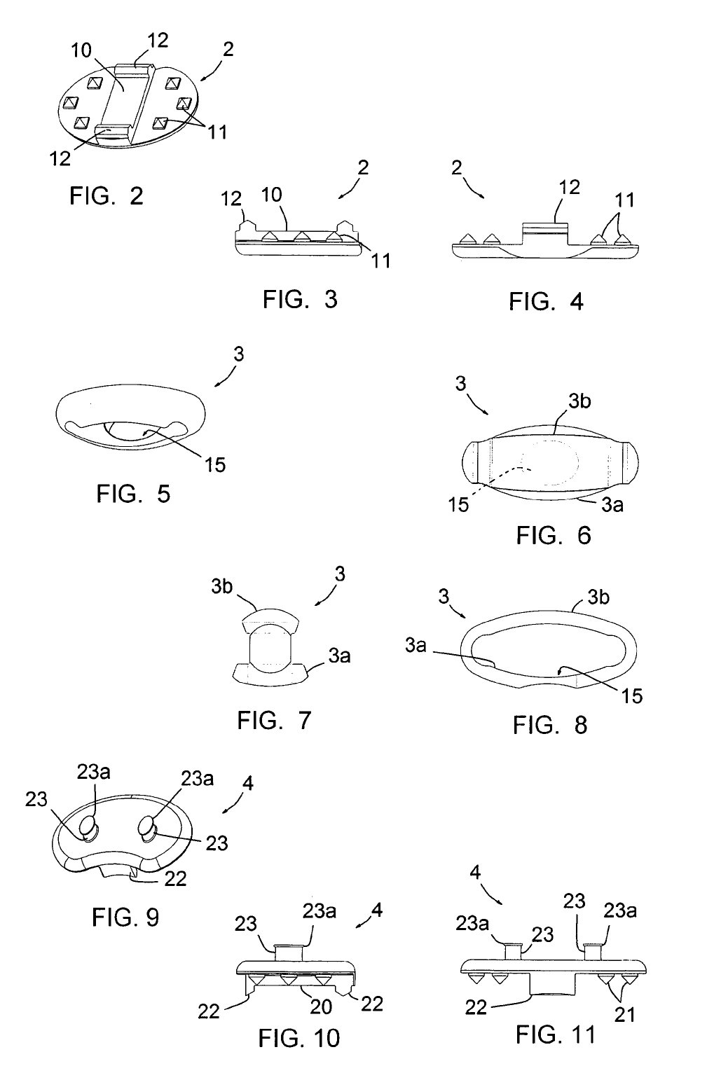

FIGS. 2 to 4 are, respectively, views in perspective, from the side via the anteroposterior plane and from the side in the frontal plane, of an upper plate, i.e. intended to be attached to the overlying vertebra;

FIGS. 5 to 8 are, respectively, views in perspective, from above, from the side via the frontal plane and from the side in the anteroposterior plane of an elastic ring according to a first embodiment;

FIGS. 9 to 11 are, respectively, views in perspective, from the side via the anteroposterior plane and from the side in the frontal plane, of a lower plate, i.e. intended to be attached to the overlying vertebra, according to a first embodiment;

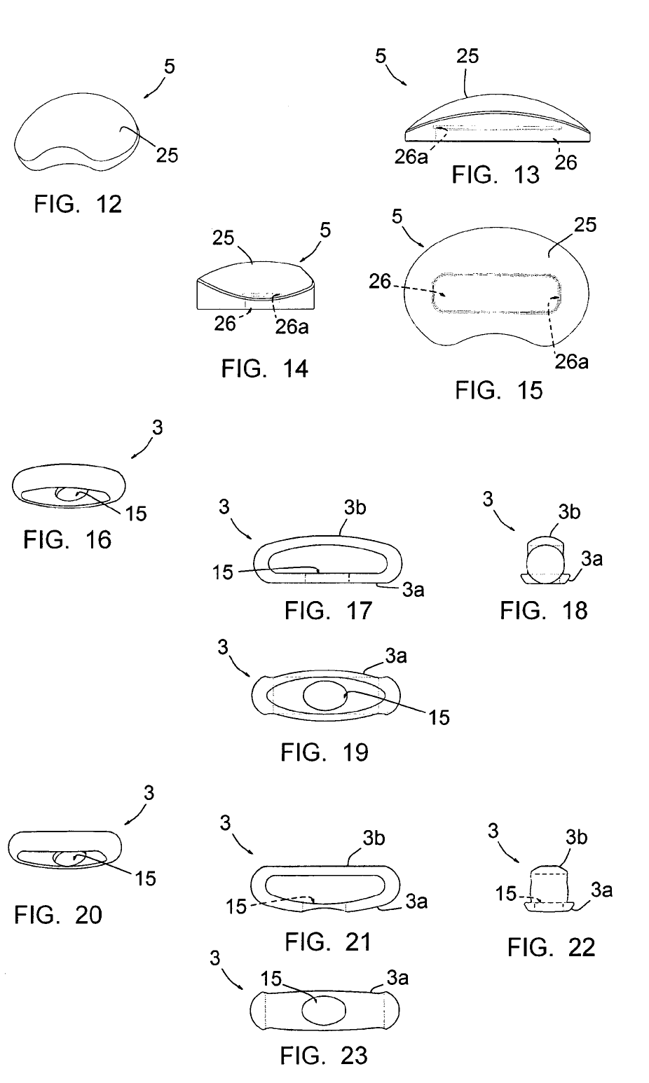

FIGS. 12 to 15 are, respectively, views in perspective, from the side via the frontal plane, from the side in the anteroposterior plane and from above, of an intermediate plate;

FIGS. 16 to 19 are, respectively, views in perspective, from the side in the anteroposterior plane, from the side in the frontal plane and from below, of an elastic ring according to a second embodiment;

FIGS. 20 to 23 are, respectively, views in perspective, from the side in the anteroposterior plane, from the side in the frontal plane and from below, of an elastic ring according to a third embodiment;

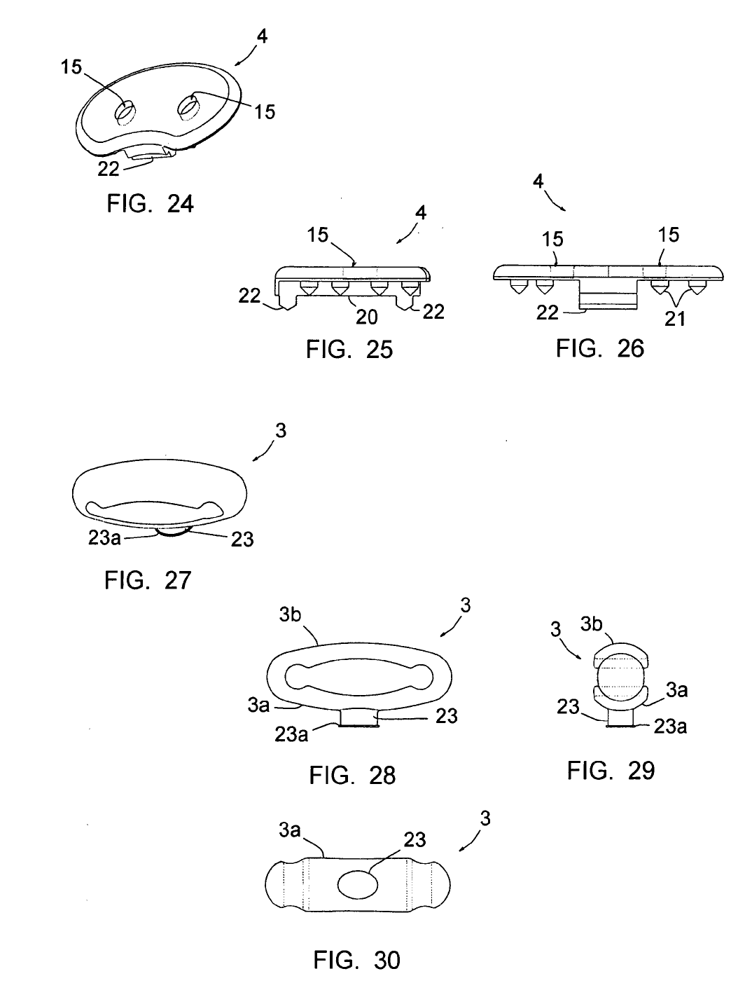

FIGS. 24 to 26 are, respectively, views in perspective, from the side via the anteroposterior plane and from the side in the frontal plane, of a lower plate according to a second embodiment, and

FIGS. 27 to 30 are, respectively, views in perspective, from the side in the anteroposterior plane, from the side in the frontal plane and from below, of an elastic ring according to a fourth embodiment, intended to be used with the lower plate illustrated in FIGS. 24 to 26.

FIG. 1 represents two vertebrae 100, 101, i.e. one overlying vertebra 100 and one underlying vertebra 101, between which a vertebral disk prosthesis 1 according to the invention has been fitted.

This prosthesis 1 comprises an upper plate 2, i.e. intended to be attached to the overlying vertebra 100, two elastic rings 3 and a lower plate 4, i.e. intended to be attached to the underlying vertebra 101.

This prosthesis 1 is formed from a component assembly comprising, in addition to the plates 2 and 4 and the rings 3, an intermediate plate 5.

The plates 2 and 4, the rings 3 and intermediate plate 5 are made of metallic material, particularly titanium alloy.

FIGS. 2 to 4 more specifically illustrate the upper plate 2, which comprises, on its side intended to be anchored to the bone, a bone anchorage median anteroposterior rib 10 and two lateral series of three bone anchorage blocks 11. The median rib 10 displays transversal swellings 12 at the anteroposterior ends thereof. Each block 11 has a pyramidal shape. The opposite side of the plate 2 is plane and smooth.

With reference to FIGS. 5 to 8, each ring 3 has an elliptical shape viewed in the plane of the opening thereof (see FIG. 8) and extended and rounded lower 3a and upper longitudinal portions 3b in the frontal plane (see FIG. 7). The lower longitudinal portion 3a comprises an oval opening 15 wherein the length is parallel to the length of the ring 3.

Each ring 3 is elastically compressible along a direction perpendicular to its length.

With reference to FIGS. 9 to 11, the lower plate 4 comprises, on one side, a bone anchorage median anteroposterior rib 20, two lateral series of three anchoring blocks 21 and swellings 22, respectively identical to the rib 10, blocks 11 and swellings 12 of the plate 2. On its opposite side, the plate 4 comprises two oval blocks 23 attached thereto, arranged at a distance from each other in the frontal plane and substantially at equidistance from the median anteroposterior plane of the plate 4. Each block 23 displays, on its free end, a projecting peripheral rim 23a.

As can be understood with reference to FIG. 1, each of the rings 3 is intended to be inserted on one of the blocks 23 via the opening 15 comprised therein, being arranged substantially parallel with the anteroposterior plane of the prosthesis 1, and each of these rings 3 is intended to bear against the lower plate 4 and receive the upper plate 2.

The rings 3, in combination with these plates 2 and 4, enable a perfect restoration of the anatomical position of both vertebrae 100, 101 and a perfect restoration of the anatomical movement of said vertebrae, with a damping effect of the weight borne thereon by the overlying vertebra 100.

The rounded longitudinal portions 3a, 3b of each ring 3 enable a rolling movement of the plates 2 and 4 (particularly of plate 2) on the ring, according to the movement of the vertebra 100 with respect to the vertebra 101. The respective dimensions of the blocks 23 and the openings 15 are such that a slight angular clearance of the rings 3 is possible in the frontal plane and/or the anteroposterior plane, with respect to the lower plate 4.

The intermediate plate 5 is formed by a metallic material part, particularly made of titanium alloy. This plate 5 displays a convex rounded side 25, intended to cooperate with the plate 2, and a plane opposite side, intended to bear against the plate 4. The plate 5 also comprises a transversal recess 26 opening into said plane face, wherein the base is bordered by a peripheral groove 26a.

This recess 26 is designed such that the plate 5 can be inserted closely on the blocks 23 of the plate 4, until the click and ratchet of the peripheral rims 23a of the blocks 23 in the groove 26a of the recess 26.

The component assembly 2 to 5 is used to form either a “dynamic” prosthesis 1 comprising the two rings 3 mentioned above, or a prosthesis with no damping effect comprising said intermediate plate 5, by means of simple adaptation on the plate 4, either of the two rings 3, or the intermediate plate 5. This component assembly thus makes it possible to obtain a vertebral disk prosthesis perfectly adaptable to the different situations liable to occur, particularly during the procedure.

FIGS. 16 to 19, on one hand, and 20 to 23, on the other, demonstrate that each ring 3 may display a rectilinear lower 3a or supper longitudinal portion 3b.

FIGS. 24 to 27 demonstrate that the lower plate 4 may comprise two oval cavities 15 instead of the blocks 23, and that, in this case, each ring 3 comprises a block 23 projecting from its lower longitudinal portions 3a, that can be inserted into any of the cavities 15.

As can be seen from the above description, the invention provides a vertebral disk prosthesis and a component assembly for the formation of a vertebral disk prosthesis with the following decisive advantages:
enable a perfect restoration of the anatomical position of both vertebrae treated and a perfect restoration of the anatomical movements of said vertebrae, with a damping effect of the weight borne thereon by the overlying vertebra, and
make it possible to form either a “dynamic” prosthesis comprising the two rings mentioned above, or a prosthesis with no damping effect comprising said intermediate plate, by means of the simple adaptation on one of the plates, either of the two rings, or the intermediate plate. The component assembly thus makes it possible to obtain a vertebral disk prosthesis perfectly adaptable to the different situations liable to arise, particularly during the procedure.

It goes without saying that the invention is not restricted to the embodiment described above as an example but is extended to any embodiments covered by the claims appended hereto.

1. Vertebral disk prosthesis (1), characterized in that it comprises:
an upper plate (2), provided, on one side, with anchoring means (10, 11) to the lower side of the overlying vertebra (100) to be treated;
a lower plate (4), provided, on one side, with anchoring means (20, 21) to the upper side of the underlying vertebra (101) to be treated;
two assemblies (23, 15) forming first installation means, wherein each is attached to the upper plate (2) or the lower plate (4), both assemblies (23, 15) being arranged at a distance from each other in the frontal plane and substantially at equidistance from the median anteroposterior plane of the plate (4); and
two elongated elastic rings (3), perpendicularly compressible to their lengths, and each comprising second installation means (15, 23) on a longitudinal portion (3a), each of these rings (3) being intended to be installed on one of said assemblies (23, 15) forming the first installation means by means of said second installation means (15, 23), being arranged substantially parallel with the anteroposterior plane of the prosthesis (1), and each of these rings (3) being intended to bear against the lower plate (4) and receive the upper plate (2).

2. Prosthesis (1) according to claim 1, characterized in that the rings (3) are made of metal, particularly titanium alloy.

3. Prosthesis (1) according to claim 1, characterized in that each ring (3) comprises two longitudinal portions (3a, 3b), and in that at least one of the longitudinal portions (3a, 3b) of each ring (3) is rounded in the frontal plane in order to enable a rolling movement on the ring (3) of the plate (2, 4) against which said longitudinal section (3a, 3b) bears, according to the movement of the overlying vertebra (100) with respect to the underlying vertebra (101).

4. Prosthesis (1) according to claim 1, characterized in that each ring (3) has, viewed in the plane of the opening thereof, an elliptical shape.

5. Prosthesis (1) according to claim 1, characterized in that each ring (3) displays a plane longitudinal portion (3a, 3b), either the lower portion or the upper portion.

6. Prosthesis (1) according to claim 1, characterized in that said first installation means comprise an oval shaped block (23) and said second installation means comprise a corresponding oval opening or cavity (15).

7. Prosthesis (1) according to claim 6, characterized in that each block (23) has a projecting peripheral rim (23a) at its free end, forming a retention means of the block (23) in the corresponding opening or cavity (15).

8. Component assembly for the formation of a vertebral disk prosthesis (1), characterized in that it comprises at least one upper plate (2), at least one lower plate (4) and at least two rings (3) according to claim 1, and at least one intermediate plate (5) with a non-elastic structure, each intermediate plate (5) having a convex rounded side (25) and third installation means (26) on the opposite side thereof, said third installation means (26) being able to cooperate with said first installation means (23) to enable an installation of said intermediate plate (5) on either the upper or lower plate (2, 4).

9. Component assembly according to claim 8, characterized in that it comprises several upper plates (2) and/or lower plates (4) and/or several pairs of elastic rings (3) and/or several intermediate plates (5) of different sizes.

10. Component assembly according to claim 8, characterized in that said third installation means consist of a recess or blocks (26) intended to cooperate with, respectively, blocks (23) or corresponding cavities of one of the plates (4).

11. Prosthesis (1) according to claim 2, characterized in that each ring (3) comprises two longitudinal portions (3a, 3b), and in that at least one of the longitudinal portions (3a, 3b) of each ring (3) is rounded in the frontal plane in order to enable a rolling movement on the ring (3) of the plate (2, 4) against which said longitudinal section (3a, 3b) bears, according to the movement of the overlying vertebra (100) with respect to the underlying vertebra (101).

12. Prosthesis (1) according to claim 2, characterized in that each ring (3) has, viewed in the plane of the opening thereof, an elliptical shape.

13. Prosthesis (1) according to claim 3, characterized in that each ring (3) has, viewed in the plane of the opening thereof, an elliptical shape.

14. Prosthesis (1) according to claim 2, characterized in that each ring (3) displays a plane longitudinal portion (3a, 3b), either the lower portion or the upper portion.

15. Prosthesis (1) according to claim 2, characterized in that each ring (3) displays a plane longitudinal portion (3a, 3b), either the lower portion or the upper portion.

16. Prosthesis (1) according to claim 2, characterized in that said first installation means comprise an oval shaped block (23) and said second installation means comprise a corresponding oval opening or cavity (15).

17. Prosthesis (1) according to claim 3, characterized in that said first installation means comprise an oval shaped block (23) and said second installation means comprise a corresponding oval opening or cavity (15).

18. Prosthesis (1) according to claim 4, characterized in that said first installation means comprise an oval shaped block (23) and said second installation means comprise a corresponding oval opening or cavity (15).

19. Prosthesis (1) according to claim 5, characterized in that said first installation means comprise an oval shaped block (23) and said second installation means comprise a corresponding oval opening or cavity (15).

20. Component assembly for the formation of a vertebral disk prosthesis (1), characterized in that it comprises at least one upper plate (2), at least one lower plate (4) and at least two rings (3) according to claim 2, and at least one intermediate plate (5) with a non-elastic structure, each intermediate plate (5) having a convex rounded side (25) and third installation means (26) on the opposite side thereof, said third installation means (26) being able to cooperate with said first installation means (23) to enable an installation of said intermediate plate (5) on either the upper or lower plate (2, 4).
